(19) United States
(12) Patent Application Publication
(10) Pub. No.: US 2021/0401428 A1
(43) Pub. Date: Dec. 30, 2021

(54) DEVICES AND METHODS FOR AUTOMATIC SUTURE

(71) Applicant: Health Research, Inc., Buffalo, NY (US)

(72) Inventors: Khurshid GURU, East Amherst, NY (US); Alwareed ABDELLATIF, Buffalo, NY (US)

(21) Appl. No.: 17/290,363

(22) PCT Filed: Oct. 30, 2019
(86) PCT No.: PCT/US19/58974

§ 371 Date: Apr. 30, 2021

Related U.S. Application Data
(60) Provisional application No. 62/752,812, filed on Oct. 30, 2018.
(60) Provisional application No. 62/909,539, filed on Oct. 2, 2019.

(51) Int. Cl. A61B 17/06

(52) U.S. Cl. CPC A61B 17/06066; A61B 17/06004; A61B 2017/00398; A61B 2017/06042; A61B 2017/06052; A61B 2017/06076

(57) ABSTRACT

A method includes engaging a clamp over a length of tissue to be sutured. A helical needle is advanced along a length of the clamp such that the helical needle passes through the tissue to be sutured. A leading end of a suturing thread is attached at a tip of the helical needle. The leading end of the suturing thread is captured. The helical needle is retracted back through the tissue so as to leave the thread in place. The clamp is disengaged from the tissue. A device for automatic suturing includes a clamp configured to be positioned over a length of tissues to be sutured. A helical needle is configured to be advanced and retracted along a length of the clamp. The needle has a tip. A suturing thread having a leading end is attached to the tip of the helical needle.

### CROSS-REFERENCE TO RELATED APPLICATIONS

This application claims priority to U.S. Provisional Application No. 62/752,812, filed on Oct. 30, 2018, now pending, and U.S. Provisional Application No. 62/909,539, filed Oct. 2, 2019, now pending, the disclosures of which are incorporated herein by reference.

### FIELD OF THE DISCLOSURE

The present disclosure relates to suturing, and in particular for suturing during a minimally-invasive procedure.

### BRIEF SUMMARY OF THE DISCLOSURE

In a first aspect, the present disclosure provides a method for automatic suturing. The method includes engaging a clamp over a length of tissue to be sutured. In some embodiments, the clamp may be locked in place over the tissue (e.g., over the length of tissue). A helical needle is advanced along a length of the clamp such that the helical needle passes through the tissue to be sutured. A leading end of a suturing thread is attached at a tip of the helical needle. The leading end of the suturing thread is captured, for example, when the tip of the helical needle is advanced to an end position. In some embodiments, a removable tip of the needle is captured with the leading end of the suturing thread. In such embodiments, the helical needle is retracted without the tip. The helical needle is retracted back through the tissue so as to leave the thread in place. The method includes disengaging the clamp from the tissue.

The leading end of the thread may be fixed to form one or more sutures. Similarly, a trailing end of the suturing thread may be fixed to form one or more suture. In some embodiments, both the leading end and the trailing end are fixed to form one or more suture. For example, sutures may be formed at each of the leading end and the trailing end. The leading end of the suturing thread and/or the trailing end of the suturing thread may be fixed using knots (e.g., by knotting the ends).

In some embodiments, the method may include cutting the leading end of the suturing thread so as to separate the tip of the helical needle from a thread portion to remain in place following retraction of the helical needle (e.g., a thread portion forming a suture).

The helical needle may be advanced and/or retracted by an electric motor. The helical needle may be advanced in single rotation increments. The helical needle may be advanced in increments less than a full rotation. The helical needle may be advanced in increments greater than a full rotation. The helical needle may be advanced to the end position.

In another aspect, the present disclosure may be embodied as a device for automatic suturing. The device comprises a clamp. The clamp is configured to be positioned over a length of tissues to be sutured. A lock may be provided for reversibly locking the clamp in a closed position. A helical needle is configured to be advanced and retracted along a length of the clamp. For example, the helical needle may be advanced along an interior portion of the clamp. In another example, the helical needle may be advanced over an exterior portion of the clamp. The needle has a tip, and a suturing thread having a leading end is attached to the tip of the helical needle. The needle may be hollow and the thread may be disposed through the needle.

The device may include a trap configured to capture a leading end of the thread once the helical needle has been advanced along at least a portion of the length of the clamp. The tip of the needle may be removable. The trap may be configured to capture the tip of the needle with the leading end of the thread.

The clamp may include a helical guide configured to guide the helical needle during advancement and retraction. The guide may be configured on an interior portion of the clamp. The device may further include at least one cutter for severing the suturing thread.

### DESCRIPTION OF THE DRAWINGS

For a fuller understanding of the nature and objects of the disclosure, reference should be made to the following detailed description taken in conjunction with the accompanying drawings, in which:

FIG. 1 is a partial cutaway of a device according to an embodiment of the present disclosure;

FIG. 2 is an exploded view of a device according to another embodiment of the present disclosure;

FIG. 3 shows a needle according to an embodiment of the present disclosure

FIG. 4 shows a detail view of a needle with a removable tip;

FIG. 5 shows a needle according to another embodiment of the present disclosure;

FIG. 6 is a chart showing a method according to another embodiment of the present disclosure;

FIG. 7A shows a device according to the present disclosure having a needle and a clamp in the open position;

FIG. 7B shows the device of FIG. 1 with the clamp in the closed position;

FIG. 7C shows the device of FIGS. 1 and 2 with the needle in a partially advanced position; and

FIG. 7D shows the device of FIGS. 1-3 with the needle further advanced.

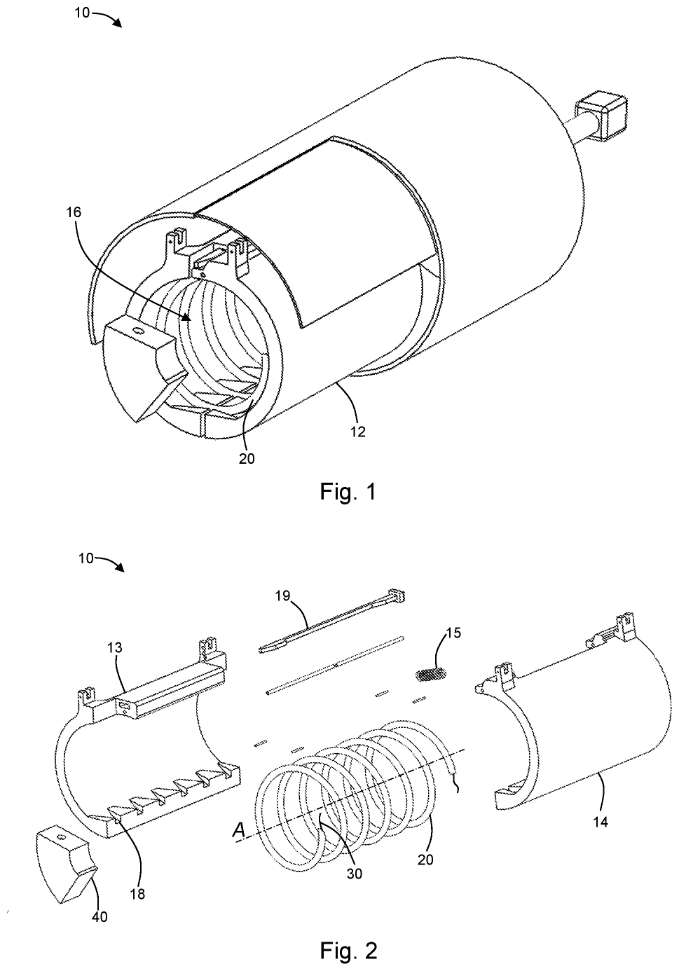

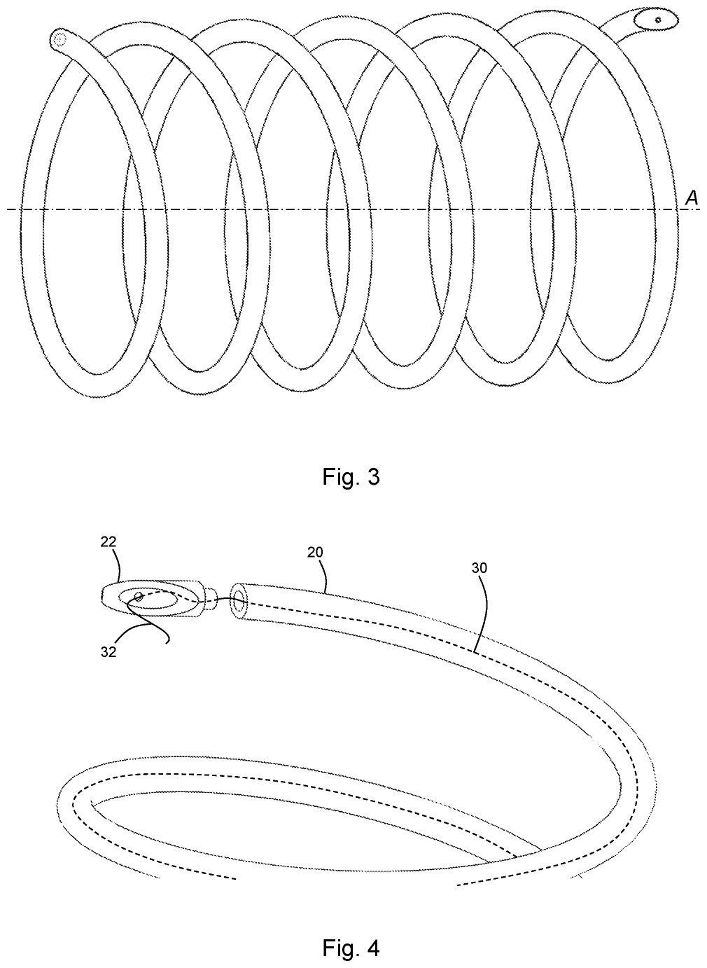

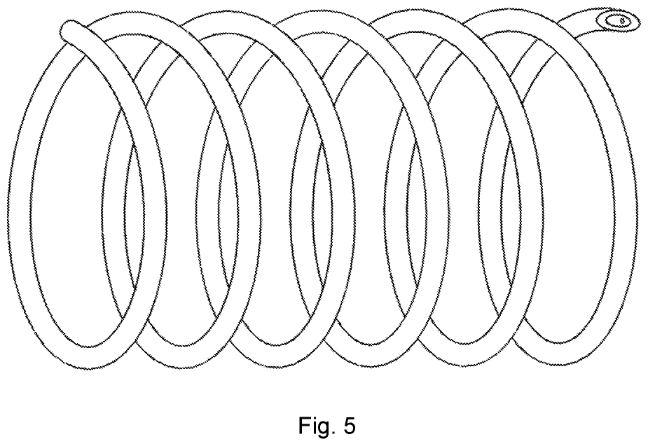

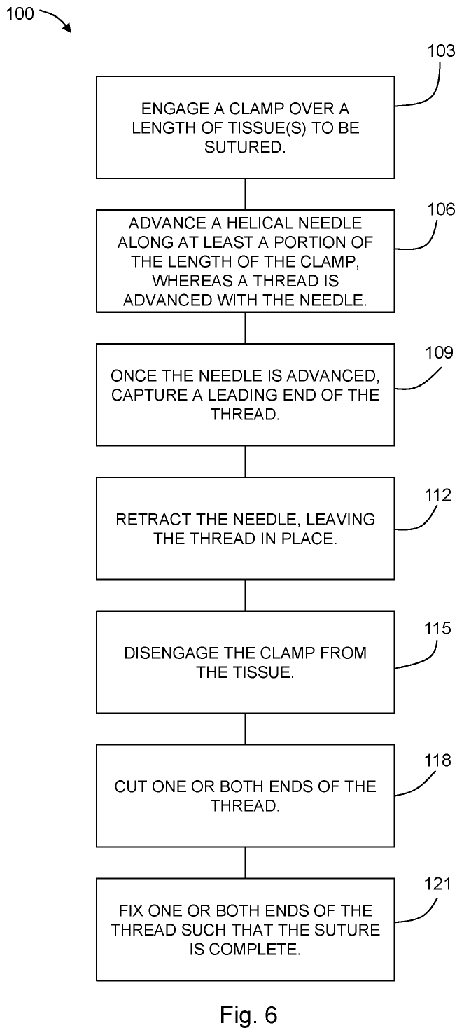

### DETAILED DESCRIPTION OF THE DISCLOSURE

In a first aspect, the present disclosure may be embodied as a method 100 for automatic suturing. For example, the method 100 may be used to create a running suture over a length of tissue (or tissues). A clamp is engaged 103 over a length of tissue (or tissues) to be sutured. For example, where bowel may need to be sutured closed over a length of the bowel, the clamp is positioned over the length of the bowel such that the two tissue portions to be sutured together are within the clamp and engaged 103 to retain the tissue therein. The clamp may be engaged 103 by a lock configured to prevent the clamp from opening.

A helical needle is advanced 106 along at least a portion of a length of the clamp. In some embodiments, the needle is advanced through the clamp such that the needle follows a helical guide in the clamp. In other embodiments, the needle is advanced over the clamp with or without a guide or guides. The clamp and the helical needle may be of any configuration as defined below. The helical needle is advanced 106 in a corkscrew-like fashion—i.e., advanced by rotating the needle about a central longitudinal axis of the helix shape. By advancing along at least a portion of the length of the clamp, the needle passes through the tissue(s) to be sutured. Based on the clamp and needle configuration and/or the manner of clamping the tissue(s) in the clamp, the needle may pass through the tissue(s) once with each rotation of the needle or twice with each rotation of the needle. The needle may be advanced by an actuator which is controlled by a surgeon. For example, each time the actuator is triggered (e.g., squeezing a trigger, depressing a button, toggling a switch, depressing a foot pedal, etc.), the needle may advance by one rotation and may puncture the tissue(s) one or two times.

In some embodiments, the needle is a hollow needle and includes a thread (a suturing thread) disposed at least partially through the hollow interior of the needle. In this way, the suturing thread is carried through the clamp and tissue by the helical needle. In some embodiments, the suturing thread is outside of the needle and attached to a tip portion of the needle. In this way, the suturing thread is pulled through the tissue by needle as the needle is advanced 106 through the clamp. In some embodiments, the needle is advanced 106 one rotation at a time (i.e., with each actuation of the device). In some embodiments, actuating the device may advance the needle by less than one rotation. For example, the actuator advances the needle by half of a rotation, a third of a rotation, a quarter of a rotation, or any other rotation less than a full rotation. As such, the tissue may be punctured by the needle one or two times for each actuation of the device or not punctured at all by actuation. In other embodiments, the needle is advanced 106 by more than one rotation at a time. For example, a surgeon may cause the needle to advance, and such advancement may continue until the needle has travelled through its entire path through the clamp. In some embodiments, the advancement 106 of the needle may occur only while activated by an operator. For example, a surgeon may squeeze a trigger to cause the needle to advance and the needle may continue to advance until the surgeon releases the trigger. The needle may advance at a selectively variable rate. For example, the advancement rate may be proportional to a position of an actuator (e.g., variable trigger, foot pedal position, etc.) In another example, the advancement rate is determined according to a control that is separate from the actuator such as, for example, a multi-setting switch, dial, rotatable knob, etc. Other advancement schemes will be apparent in light of the present disclosure.

A leading end of the suturing thread is captured 109 once the needle is advanced 106 along the clamp. For example, the suturing thread may be captured when the tip of the helical needle is advanced to an end position. The end location may be at a distal end of the clamp. In some embodiments, the end location may be at a position spaced apart from the distal end of the clamp. In some embodiments, the end position may be selected by a user according to the application. For example, in some embodiments as further described below, a trap is positioned on the clamp such that a tip of the needle is proximate to or disposed within the trap at a fully-advanced position. The trap captures 109 at least a portion of the suturing thread. In some embodiments, the tip of the needle is removable, and the tip is captured 109 along with the at least a portion of the suturing thread. In other embodiments, the suturing thread is captured manually by an operator (for example, a surgeon) using a separate device.

Once the suturing thread is captured 109, the needle is retracted 112 back along the length clamp. For example, the needle is retracted 112 using an opposite corkscrew movement from the advancing movement—i.e., by oppositely-rotating the needle about the central longitudinal axis of the helix shape. Because at least a portion of the suturing thread is captured 109, the suturing thread is left in place during retraction 112 of the needle. By left in place, it is intended that the suturing thread remain disposed through the tissue(s) at the locations where the needle passed through the tissue(s). In embodiments where the suturing thread is disposed through a hollow portion of the needle, the needle is retracted 112 by passing over the suturing thread (the suturing thread slides through the hollow portion, thereby remaining in place once the needle is retracted).

The clamp is disengaged 115 from the tissue. In this way, the needle has been retracted and all components may be removed from the body of the individual. In some embodiments, the suturing thread may be cut 118 at the leading end and/or a trailing end such that the clamp and needle may be removed while leaving the suturing thread in place.

The leading end of the suturing thread and/or the trailing end of the suturing thread are fixed to complete the suture. In some embodiments, one or both of the ends are fixed 121 by knotting the thread (securing the thread to itself). In some embodiments, the suturing thread is knotted such that one or both ends cannot pull through the tissue. In some embodiments, the suturing thread is configured to swell to a thicker diameter (in this case, thicker means a diameter greater than an initial diameter of the suturing thread) thereby retaining the suturing thread in the tissue. Such threads are known in the art. Before fixation of one or both ends, the suturing thread may be adjusted such that the suture is tightened as desired. One or both ends of the suturing thread may be fixed before or after disengagement 115 of the clamp.

In some embodiments, the helical needle is advanced 106 and/or retracted 112 using an electric motor. In some embodiments, the helical needle is advanced 106 and/or retracted 112 using by way of a manual operation (e.g., a hand crank, etc.)

In another aspect, the present disclosure may be embodied as a device for automatic suturing. The device 10 includes a clamp 12. A guide 18 may be disposed on an interior portion 16 of the clamp 12. In an exemplary embodiment, the clamp 12 includes a first side portion 13 and a second side portion 14, each hingedly connected to the other. A spring 15 may cause the first and second side portions 13,14 to be biased toward each other—i.e., biased to a “closed” state of the clamp 12. Such a clamp 12 is configured to be opened and positioned over a length of tissue. The clamp 12 may be engaged by allowing the clamp to return to the closed position (e.g., partially closed as the tissue(s) now cause some separation at an opening of the clamp 12). In other embodiments, the clamp is not spring biased and the clamp is operably closable by a user. The clamp 12 may further include a lock 19 configured to prevent the clamp 12 from inadvertent opening.

A guide 18 may be provided on an interior portion 16 of the clamp 12. The device further comprises a helical needle 20. The needle 20 and helical guide 18 are configured such that the needle 20 can be advanced along a length of the clamp by rotating the needle 20 about a central longitudinal axis A of the needle 20 (and guide 18). In this way, the needle 20 will pierce the tissue one or more times as it rotates through the helical guide 18. The needle 20 may have a gauge of, for example, between 20 G and 10 G (Birmingham gauge; approximately 0.91-3.4 mm, outer diameter), inclusive, but may be smaller or larger as desired for the application. The overall diameter of the helix shape may be, for example, between 0.25-2.0 in (approximately 6.35-50.8 mm), inclusive, but may be smaller or larger as desired for a particular application. The helical guide 18 of the clamp 12 has a helix diameter which corresponds to and accommodates the needle 20 selected for a particular device.

The device 10 further includes a suturing thread 30 for suturing the tissue. The suturing thread has a leading end 32 which may be attached to the needle 20 (see, e.g., FIG. 4). In some embodiments, the leading end 32 is attached to a tip 22 of the needle 20. In an example, the suturing thread 30 may be attached by way of a spring-loaded, multi-prong grasper as is known in the art. In other embodiments, the suturing thread 30 is attached by way of being tied to an eye of the needle 20. Other techniques for attachment will be apparent in light of the present disclosure. In some embodiments, the needle 20 is hollow and the suturing thread 30 is disposed through the hollow portion of the needle 20 emerging at the tip 22. In other embodiments, the suturing thread may be generally external to the needle and attached at the tip. The needle 20 and suturing thread 30 are configured such that as the needle 20 is advanced along a length of the clamp—for example, through the guide 18 of the clamp 12—and through portions of the tissue(s) held by the clamp 12, the suturing thread 30 is carried through the tissue as well.

The number of sutures through the held tissue will generally match the number of helixes. For example, if a helix-shaped needle 20 includes 24 helixes, there will generally be a result of 24 sutures (assuming tissue was properly placed in the clamp 12, etc.) Note that the resulting suture is not 24 individual sutures, but a running suture or “continuous stitch”—i.e., a continuous suture having only the end stitches tied off. The needle 20 may have, for example, between 12 and 24 helixes, inclusive, but may have more helixes or fewer helixes. The needle 20 may have a length of, for example, between 3 inches and 6 inches (approximately 76.2-152.4 mm), inclusive, but may be longer or shorter.

The device 10 may include a trap 40 configured to engage with the tip 22 of the needle 20 when the needle 20 has been completely advanced through the clamp 12. By engage the needle 20, it is intended that the trap 40 is configured to capture the suturing thread 30—e.g., at least a portion of the thread, for example, at or near the leading end 32 of the suturing thread 30. In some embodiments, the trap 40 is configured such that the tip 22 of the needle 20 enters the trap 40. In some embodiments, the tip 22 is removable, and the trap 40 may be configured to remove and retain the tip 32 (along with at least a portion of the leading end 32 of the thread) within the trap 40. By capturing the suturing thread 30, the helical needle 20 may be retracted through the clamp 12 while leaving the suturing thread 30 in place.

In some embodiments, the device 10 may further include one or more cutters to sever the suturing thread 30 at the leading end 32 and/or a trailing end 34. In this way, the device 10 may be removed from the body while leaving the suturing thread 30 in place through the tissue(s).

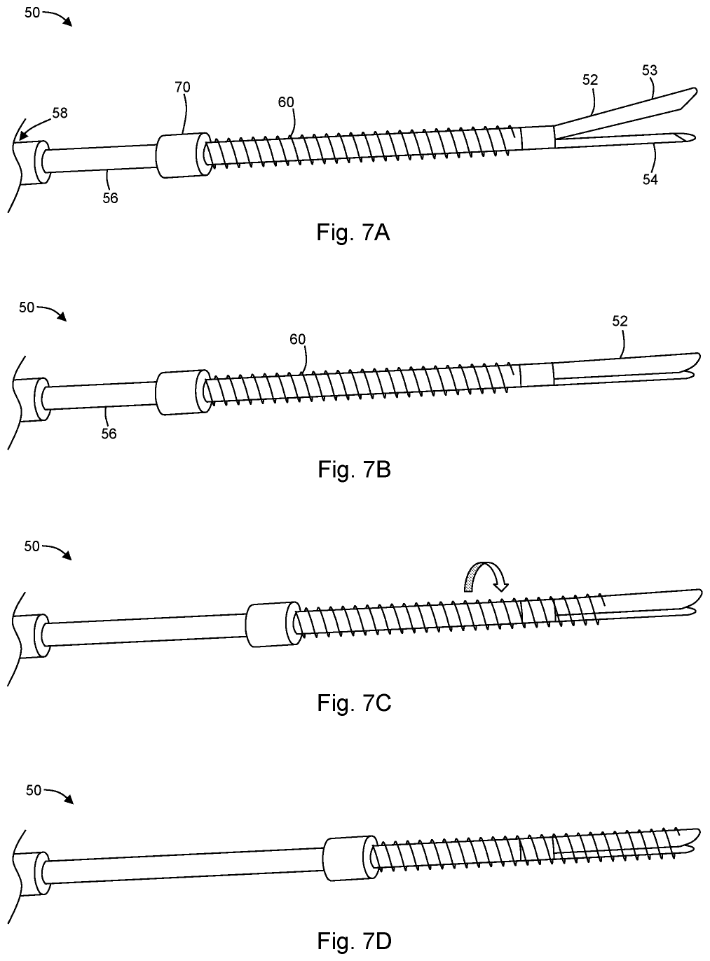

In another embodiment, the device may be configured such that the helical needle is advanced and retracted over (e.g., external to) the clamp. For example, with reference to FIGS. 7A-7D, a device 50 includes a clamp 52. The clamp 52 may be attached at an end of a shaft 56. In an exemplary embodiment, the clamp 52 includes a first side portion 53 and a second side portion 54, each hingedly connected to the other. The first and second side portions 53,54 may be biased toward each other—i.e., biased to a “closed” state of the clamp 52. Such a clamp 52 is configured to be opened and positioned over a length of tissue. The clamp 52 may be engaged by allowing the clamp to return to the closed position (e.g., partially closed as the tissue(s) now cause some separation at an opening of the clamp 52). In other embodiments, the clamp is not spring biased and the clamp is operably closable by a user. The clamp may further include a lock configured to prevent the clamp from inadvertent opening.

The device 52 further comprises a helical needle 60. The helical needle 60 can be advanced along a length of the clamp by rotating the helical needle 60 about a central longitudinal axis of the helical needle 60. In this way, the needle 60 will pierce the tissue one or more times as it rotates along the length of the clamp 52. In some embodiments, the helical needle 60 may be disposed over the shaft 56 when in a retracted position (FIGS. 7A and 7B). In this way, the clamp 52 may be opened (FIG. 7A) and closed (FIG. 7B) over the tissue(s). When the clamp 52 is closed (clamped) on the tissue(s) to be sutured, the helical needle 60 may be advanced along a length of the clamp 52 (see, e.g., FIGS. 7C and 7D)—i.e., along the longitudinal length of the clamp 52. For example, the helical needle may be advanced over an exterior of the clamp. The shaft and/or the clamp may include a guide or guides (not shown) to guide the helical needle during advancement and retraction. The device may further include a sheath configured to enclose at least a portion of the helical needle when in a retracted position.

The device may include an operable end for operation by a user. In some embodiments, the device 50 may include a coupler (generally at 58) for coupling to a portion for operation by a user (for example, a control end). In some embodiments, the device 50 includes a motor 70, such as, for example, an electric motor, operably coupled to the helical needle 60. As such, the motor 70 may rotate the helical needle 60. In some embodiments, such as, for example, some embodiments having a guide or guides, rotation of the needle also causes advancement or retraction of the needle along its length. In other embodiments, the motor 70 may also provide translation to advance and retract the helical needle along its length. In other embodiments, rotation and or translational movement of the helical needle is provided in other ways, such as, for example, provided external from the device.

### Further Examples

Example 1. A method for automatic suturing, comprising: engaging a clamp over a length of tissue to be sutured; advancing a helical needle along a length of the clamp such that the helical needle passes through the tissue to be sutured, and wherein a leading end of a suturing thread is attached at a tip of the helical needle; capturing the leading end of the suturing thread; retracting the helical needle back through the tissue so as to leave the suturing thread in place; disengaging the clamp from the tissue.

Example 2. The method of Example 1, further comprising fixing the leading end of the suturing thread and/or a trailing end of the suturing thread to form one or more sutures.

Example 3. The method of Example 2, wherein the leading end of the suturing thread and/or the trailing end of the suturing thread are fixed by knotting.

Example 4. The method of any one of Examples 1-3, wherein the clamp is locked in place over the tissue.

Example 5. The method of any one of Examples 1-4, wherein a removable tip of the helical needle is captured with the leading end of the suturing thread and the helical needle is retracted without the tip.

Example 6. The method of any one of Examples 1-5, further comprising cutting the leading end of the suturing thread so as to separate the tip of the helical needle from a thread portion forming the suture.

Example 7. The method of any one of Examples 1-6, wherein the helical needle is advanced by an electric motor.

Example 8. The method of any one of Examples 1-7, wherein the helical needle is advanced in single rotation increments.

Example 9. A device for automatic suturing, comprising: a clamp configured to be positioned over a length of tissue to be sutured; a helical needle configured to be advanced and retracted along a length of the clamp, the helical needle having a tip; and a suturing thread having a leading end attached to the tip of the helical needle.

Example 10. The device of Example 9, further comprising a trap configured to capture a leading end of the suturing thread once the helical needle has been advanced along at least a portion of the length of the clamp.

Example 11. The device of any one of Examples 9-10, wherein the clamp includes a helical guide configured to guide the helical needle during advancement and retraction.

Example 12. The device of Example 11, wherein the helical guide is on an interior portion of the clamp.

Example 13. The device of any one of Examples 9-12, wherein the helical needle is hollow and the suturing thread is disposed through the helical needle.

Example 14. The device of any one of Examples 9-13, wherein the tip of the helical needle is removable and the trap is configured to capture the tip of the helical needle with the leading end of the suturing thread.

Example 15. The device of any one of Examples 9-14, further comprising at least one cutter for severing the suturing thread.

Example 16. The device of any one of Example 9-15, further comprising a lock for reversibly locking the clamp in a closed position.

Example 17. The device of any one of Examples 9-16, wherein the helical needle is configured to be advanced along an inside portion of the clamp.

Example 18. The device of any one of Examples 9-11 and 13-16, wherein the helical needle is configured to be advanced over an exterior of the clamp.

Example 19. The device of any one of Examples 9-18, further comprising a shaft attached to the clamp, and wherein the helical needle is configured to be disposed over the shaft when in a retracted position.

Example 20. The device of any one of Examples 9-19, further a motor operably connected to the helical needle and configured to rotate the helical needle.

Although the present disclosure has been described with respect to one or more particular embodiments, it will be understood that other embodiments of the present disclosure may be made without departing from the spirit and scope of the present disclosure.

1. A method for automatic suturing, comprising:
engaging a clamp over a length of tissue to be sutured;
advancing a helical needle along a length of the clamp such that the helical needle passes through the tissue to be sutured, and wherein a leading end of a suturing thread is attached at a tip of the helical needle;
capturing the leading end of the suturing thread;
retracting the helical needle back through the tissue so as to leave the suturing thread in place;
disengaging the clamp from the tissue.

2. The method of claim 1, further comprising fixing the leading end of the suturing thread and/or a trailing end of the suturing thread to form one or more sutures.

3. The method of claim 2, wherein the leading end of the suturing thread and/or the trailing end of the suturing thread are fixed by knotting.

4. The method of claim 1, wherein the clamp is locked in place over the tissue.

5. The method of claim 1, wherein a removable tip of the helical needle is captured with the leading end of the suturing thread and the helical needle is retracted without the tip.

6. The method of claim 1, further comprising cutting the leading end of the suturing thread so as to separate the tip of the helical needle from a thread portion forming the suture.

7. The method of claim 1, wherein the helical needle is advanced by an electric motor.

8. The method of claim 1, wherein the helical needle is advanced in single rotation increments.

9. A device for automatic suturing, comprising:
a clamp configured to be positioned over a length of tissue to be sutured;
a helical needle configured to be advanced and retracted along a length of the clamp, the helical needle having a tip; and
a suturing thread having a leading end attached to the tip of the helical needle.

10. The device of claim 9, further comprising a trap configured to capture a leading end of the suturing thread once the helical needle has been advanced along at least a portion of the length of the clamp.

11. The device of claim 9, wherein the clamp includes a helical guide configured to guide the helical needle during advancement and retraction.

12. The device of claim 11, wherein the helical guide is on an interior portion of the clamp.

13. The device of claim 9, wherein the helical needle is hollow and the suturing thread is disposed through the helical needle.

14. The device of claim 10, wherein the tip of the helical needle is removable and the trap is configured to capture the tip of the helical needle with the leading end of the suturing thread.

15. The device of claim 9, further comprising at least one cutter for severing the suturing thread.

16. The device of claim 9, further comprising a lock for reversibly locking the clamp in a closed position.

17. The device of claim 9, wherein the helical needle is configured to be advanced along an inside portion of the clamp.

18. The device of claim 9, wherein the helical needle is configured to be advanced over an exterior of the clamp.

19. The device of claim 9, further comprising a shaft attached to the clamp, and wherein the helical needle is configured to be disposed over the shaft when in a retracted position.

20. The device of claim 9, further a motor operably connected to the helical needle and configured to rotate the helical needle.
